(19) United States
(12) Patent Application Publication
(10) Pub. No.: US 2010/0331676 A1
(43) Pub. Date: Dec. 30, 2010

(54) GAMMA PROBE DETECTION OF AMYLOID PLAQUE USING RADIOLABELED A-BETA BINDING COMPOUNDS

(75) Inventor: Alan P. Carpenter, Carlisle, MA (US)

Correspondence Address: PEPPER HAMILTON LLP, ONE MELLON CENTER, 50TH FLOOR, 500 GRANT STREET, PITTSBURGH, PA, 15219, US

(73) Assignee: AVID RADIOPHARMACEUTICALS, INC., Philadelphia, PA (US)

(21) Appl. No.: 12/918,455

(22) PCT Filed: Feb. 23, 2009
(86) PCT No.: PCT/US2009/034886

§ 371 Date: Aug. 19, 2010

Related U.S. Application Data
(60) Provisional application No. 61/031,809, filed on Feb. 27, 2008.

(51) Int. Cl. A61B 6/00; A61K 51/00

(52) U.S. Cl. 600/431; 424/1.61

(57) ABSTRACT

The present invention relates to a method of detecting β-amyloid peptide aggregates in the brain of an individual and kits thereto. The method includes administering to an individual an effective amount of an Aβ-binding radiopharmaceutical, waiting a period of time, measuring a gamma radiation count over an external area of the head corresponding to the cortex of the individual using a radiation detection device, and comparing the gamma radiation count with a control gamma radiation count.

### CROSS-REFERENCE TO RELATED APPLICATIONS

This application claims priority under 35 U.S.C. §119(e) to U.S. Provisional Patent Application Ser. No. 61/031,809, filed Feb. 27, 2008, the disclosure of which is incorporated by reference m its entirety.

### TECHNICAL FIELD

The invention relates generally to monitoring physiological activity in a human brain and more specifically to detecting the presence of amyloid plaque in a human brain using a radiation detection device together with a gamma-emitting radiopharmaceutical that binds to β-amyloid plaque.

### BACKGROUND OF THE INVENTION

Alzheimer's disease (AD) is a progressive neurodegenerative disorder characterized by cognitive decline, irreversible memory loss, disorientation, and language impairment. It is the most common cause of dementia in the United States. AD can strike persons as young as 40-50 years of age, but because the presence of the disease is difficult to detect without histopathological examination of brain tissue, the time of onset in living subjects is unknown. The prevalence of AD increases with age, with estimates of the affected population as high as 40% by ages 85-90.

In practice, AD is definitively diagnosed through examination of brain tissue, usually at autopsy. Postmortem examination of AD brain sections reveals abundant senile plaques (SPs) composed of amyloid-β (Aβ) peptide aggregates and neurofibrillary tangles (NFTs) formed by filaments of highly phosphorylated tau proteins.

Given the nexus between Aβ aggregates and AD, radiolabeled compounds have been developed for imaging Aβ aggregates (i.e., amyloid plaque). For instance, several radioisotopically-labeled Aβ-aggregate specific ligands are available for the imaging of amyloid plaque in a living subject using positron emission tomography (PET) or single photon emission tomography (SPECT).

Despite the potential benefits of in vivo imaging of amyloid plaque, economic challenges may be associated with the use of PET or SPECT imaging techniques as screening tools. For example, these imaging techniques require specialized imaging equipment and highly trained medical personnel to perform such imaging, resulting in high costs. Given that as many as 15 million subjects in the U.S. and more than 80 million subjects worldwide may be at risk for AD by the middle of the 21^{st} century, there is a need for low cost methods for screening subjects to identify those at an elevated risk for having amyloid plaques.

### SUMMARY OF THE INVENTION

In embodiments of the present invention, a method of detecting β-amyloid aggregates in a brain of an individual is provided that includes administering to an individual an effective amount of an Aβ-binding radiopharmaceutical, waiting a period of time, measuring a gamma radiation count over an external area of the head corresponding to the cortex of the individual using a radiation detection device, and comparing the gamma radiation count, with a control gamma radiation count.

In some embodiments, the effective amount comprises from about 0.1 to about 20 mCi of Aβ-binding radiopharmaceutical. In other embodiments, the effective amount of Aβ-binding radiopharmaceutical comprises from about 0.1 to about 10 mCi. In yet other embodiments, the effective amount comprises from about 0.1 to about 2 mCi.

The Aβ-binding radiopharmaceutical of certain embodiments includes a compound having a binding affinity of ≦100 nM for Aβ-aggregates. In some embodiments, the Aβ-binding radiopharmaceutical includes a compound having a binding affinity of about 10 nM or less.

In some embodiments of the present invention, the period of waiting time is from about 5 minutes to a time corresponding to approximately twice the radioactive half-life of the radioactive isotope of the Aβ-binding radiopharmaceutical. In other embodiments, the period of waiting time is from about 10 minutes to a time corresponding to the radioactive half-life of the radioactive isotope of the Aβ-binding radiopharmaceutical. The period of waiting time in certain embodiments is about 1 to about 60 minutes.

The control gamma radiation count of embodiments of the present invention is a gamma radiation count of a control region in the brain of the individual. In some embodiments, the control gamma radiation count is a gamma radiation count obtained over an external area of the head corresponding to the cortex of individuals from a healthy control population. In certain embodiments, the control gamma radiation count is an average or median gamma radiation count determined by repeating the gamma radiation count measurement for a population of healthy individuals and calculating the average or median counts for the control population.

The comparing step of embodiments of the method of detecting β-amyloid aggregates in a brain of an individual includes calculating a ratio of the cortical gamma radiation count for an individual to the control gamma radiation count. In some embodiments, a calculated ratio of above about 1.4 is consistent with the presence of β-amyloid peptide aggregates in the brain of the individual.

According to one embodiment of the present invention the effective amount of Aβ-binding radiopharmaceutical includes from about 0.1 to about 20 mCi, the Aβ-binding radiopharmaceutical comprises a compound having a binding affinity of ≦100 nM for Aβ-aggregates, the period of waiting time is from about 0.1 hours to a time corresponding to about the radioactive half-life of the radioisotope attached to said Aβ-binding radiopharmaceutical, and the control gamma radiation count is obtained by measuring the gamma radiation over the external area of the head corresponding to the cerebellum region of said individual using said radiation detection device.

In another embodiment of the present invention, a kit for detecting amyloid plaques in the brain of an individual is provided that includes an Aβ-binding radiopharmaceutical and instructions for using the Aβ-binding radiopharmaceutical. The instructions include a direction to administer to an individual an effective amount of an Aβ-binding radiopharmaceutical, a direction to wait a period of time, and a direction to measure a gamma radiation count over an external area of the head corresponding to the cortex of said individual using a radiation detection device. The Aβ-binding radiopharmaceutical of certain embodiments is in dosage form for intraveneous injection. In some embodiments, the kit further includes a radiation detection device and instructions for using such radiation detection device. In other embodiments, the kit further includes instructions for using a radiation detection device at a disclosed location.

### BRIEF DESCRIPTION OF THE FIGURES

FIG. 1 schematically illustrates gamma probe detection of amyloid plaques following injection of radiolabeled Aβ-binding compounds according to one embodiment of the present invention.

### DETAILED DESCRIPTION OF EXEMPLARY EMBODIMENTS

This invention is not limited to the particular compositions or methodologies described, as these may vary. All publications and references mentioned herein including all patents, patent applications, and publications, are incorporated by reference. Nothing herein is to be construed as an admission that the invention is not entitled to antedate such disclosure by virtue of prior invention.

In addition, the terminology used in the description describes particular versions or embodiments only, and is not intended to limit the scope of the present invention. Unless defined otherwise, all technical and scientific terms used herein have the same meanings as commonly understood by one of ordinary skill in the art.

As used herein, the singular forms “a”, “an” and “the” include plural reference unless the context clearly dictates otherwise.

As used herein, the terms “Aβ-binding radiopharmaceutical”, “Aβ-aggregate binding radiopharmaceutical”, and “A-Beta binding radiopharmaceutical” refer to a compound, or pharmaceutically acceptable salt thereof that binds to amyloid-β peptide aggregates or amyloid plaques and that is radiolabeled with an isotope, which decays with an emission of a gamma-ray or alternatively emits a positron that upon annilation results in two opposing 511 keV gamma rays.

As used herein, the term “about” means plus or minus 10% of the numerical value of the number with which it is being used. Therefore, about 50% means in the range of 40-60%.

“Administering” when used in conjunction with a therapeutic means to administer a therapeutic directly into or onto a target tissue or to administer a therapeutic to a patient whereby the therapeutic impacts the tissue to which it is targeted. “Administering” a composition may be accomplished, for example, by injection, infusion, or by either method in combination with other known techniques. Such combination techniques include heating, radiation and ultrasound.

As used herein, the terms “β-amyloid aggregates”, “β-amyloid peptide aggregates”, “amyloid plaques” and “Aβ aggregates” include, but me not limited to, insoluble polymers or aggregates of Aβ40 or Aβ42 peptides.

The term “binding affinity”, as used herein, refers to K_{d} (or Kᵢ versus a well-characterized competitive Aβ-binding ligand) for a radiopharmaceutical binding to β-amyloid aggregates.

In some embodiments, the term “control gamma radiation count” refers to a gamma radiation count obtained with a radiation detection device following injection of an Aβ-binding radiopharmaceutical in measurements over a brain of a healthy individual or an average or median gamma radiation count obtained for a population of such individuals. In other embodiments, the term “control gamma radiation count” refers to a gamma radiation count obtained with a radiation detection device following injection of an Aβ-binding radiopharmaceutical in measurements over a brain region of an individual that does not normally contain Aβ-aggregates, such as the cerebellum (e.g., over the back and base of the head), or an average or median obtained for a population of such individuals.

As used herein, the term “detector component” refers to an element or elements for capturing gamma rays and converting such captured gamma rays into an electrical detector output. Suitable detector components include, but are not limited to, a scintillation crystal that captures gamma rays and converts them into a light signal and a component that converts the light signal into an electrical detector output.

As used herein, the term “detector output amplification component” refers to an element or elements for boosting initial detector output, such as, but not limited to, one or more photomultiplier tubes or a photodiode array for boosting light signal, or combinations thereof.

The term “diseased tissue”, as used herein, refers to tissue or cells associated with a diseased state or exhibiting symptoms of a disease including, but not limited to, solid tumor cancers of any type, such as, but not limited to bone, lung, vascular, neuronal, colon, ovarian, breast and prostate cancer. The term “diseased tissue” may also encompass tissue of arthritic joints, such as, for example, inflamed synovial tissue.

As used herein, the term “elderly individual” refers to a human of about 50 years of age or greater.

As used herein, the term “gamma radiation count” includes, but is not limited to, a radiation count rate (counts/time) or a total radiation count acquired over a short period of time, such as, but not limited to, 1-2 minutes or less.

As used herein, the term “impacts” conveys that the present invention changes the appearance, form, characteristics and/or physical attributes of the tissue to which it is being provided, applied or administered.

The term “individual”, as used herein, refers to a living creature.

As used herein, the term “instructions” refers to any directions for using kits, including, but not limited to, written directions such as a label, pamphlet or product insert, electronic directions provided on electronic media, website or reference to a website or customer service line.

An “isotopically labeled”, “radiolabeled”, “detectable” or “detectable amyloid binding” compound, “radioligand” or “radiolabeled pharmaceutical”, as used herein, refers to a compound of the present invention where one of more atoms are replaced or substituted by an atom having an atomic mass or mass number different from the atomic mass or mass number typically found in nature (i.e., naturally occurring). Suitable radionuclides (i.e., “detectable isotopes”) that may be incorporated in the compounds of the present invention include, but are nut limited to, ¹¹C, ¹³N, ¹⁵O, ¹⁸F, ⁷⁵Br, ⁷⁶Br, ⁷⁷Br, ⁸²Br, ^{99m}Tc, ¹²³I, ¹²⁴I, ¹²⁵I, and ¹³¹I. An isotopically labeled compound need only be enriched with a detectable isotope to a degree that permits detection with a technique suitable for the particular application.

As used herein, the term “healthy individual”, “normal individual” or “normal healthy individual” refers to an individual who is not suspected to suffer from any cognitive disorder such as, but not limited to, dementia or Alzheimer's disease) and/or an individual who is not suspected to have β-amyloid peptide aggregates in the cortex of the brain such as, but not limited to, someone who is less than 50 years of age.

“Optional” or “optionally” as used herein, may be taken to mean that a subsequently described structure, event or circumstance may or may not occur and that the description of the invention includes instances where the event occurs and instances where it does not.

As used herein, the term “radiation shield with collimating aperture” is a gamma ray absorbing material such as, but not limited to, lead or tungsten that absorbs gamma radiation emanating from oblique angles from the head in relation to the gamma radiation detector device surface and that contains an opening with a diameter of, but not limited to, about 0.1 to about 2 cm, which may allow gamma rays traveling along the line (or cylinder/cone) of sight of the detector component to pass through and be detected by the radiation detection device.

The term “target”, as used herein, refers to the material for which deactivation, rupture, disruption or destruction is desired. For example, diseased tissue, pathogens, or infectious material may be a target.

As used herein, the term “therapeutic” refers to an agent utilized to treat, combat, ameliorate, impact or prevent a condition or disease in a patient.

A “therapeutically effective amount” or “effective amount” of a composition, as used herein, is a predetermined amount calculated to achieve the desired effect. In some embodiments of the present invention, the terms “therapeutically effective amount” or “effective amount” refer to the amount of Aβ-binding radiopharmaceutical(s) that results in a sufficient gamma radiation count to distinguish the gamma radiation count of an individual with Aβ aggregates in the cortex of the brain from the control gamma radiation count (such as the gamma radiation count in the cerebellum or in the cortex of a healthy individual).

The term “tissue”, as used herein, refers to any aggregation of similarly specialized cells united in the performance of a particular function.

Embodiments of the invention are directed to a low cost method of screening for the presence of amyloid plaque in a human brain using a radiation detection device together with a gamma-emitting radiopharmaceutical that binds to β-amyloid plaque.

In particular, embodiments of the present invention provide a measurement resulting in a number that can he related to an individual's risk of having Aβ plaques in the brain. Furthermore, the measurement does not mandate use of PET or SPECT imaging instrumentation. The screening methods of aspects of the present invention can be used to provide an estimate of an individuals risk of developing Alzheimer's disease (AD) or other neurodegenerative disorder associated with the presence of Aβ plaques, evaluate the progression of AD or other neurodegenerative disorder, diagnose AD or other neurodegenerative disorder, and monitor the progression of AD or other neurodegenerative disorder.

Specifically, embodiments of the invention presented herein are directed to use of a handheld or stationary radiation probe detector for non-invasive (i.e, non-surgical) screening of individuals having or at risk for acquiring AD or other neurodegenerative disorders. The screening is performed through the utilization of a radiation count rate or total radiation count measurement on the surface of the cranium over a short period of time following the administration of a radiopharmaceutical that specifically binds to Aβ aggregates in the brain.

Embodiments of the invention include a method of detecting β-amyloid peptide aggregates in the brain of an individual including the steps of: (a) administering to an individual an effective amount of a gamma-emitting Aβ-aggregate binding radiopharmaceutical; (b) waiting a period of time (i.e., the “waiting time”); (c) measuring a gamma radiation count over an external area of the head corresponding to the cortex (e.g., orbital frontal region) of the individual using a radiation detection device; and (d) comparing the gamma radiation count detected in step (c) with a control gamma radiation count.

Step (a) of the method embodied above involves the administration of an effective amount of a gamma-emitting radiopharmaceutical that binds to Aβ aggregates in the brain. The radiopharmaceutical administered in various aspects of the invention may be any radiopharmaceutical known in the art having an affinity for Aβ aggregates, and in certain embodiments, two or more radiopharmaceuticals may be administered. In some embodiments, the Aβ-aggregate binding radiopharmaceutical further includes a pharmaceutically acceptable carrier.

Step (b) of embodiments of the present invention involves waiting for a period of time following the administration of the gamma-emitting Aβ-aggregate binding radiopharmaceutical. The waiting time may be any amount of time that allows the Aβ-binding radiopharmaceutical to sufficiently clear from the blood steam of an individual being examined, localize in the brain of such individual, and bind to amyloid plaques in the brain, if present. The waiting time may vary among embodiments as a result of, for example, manner, location, and amount of Aβ-binding radiopharmaceutical administered, affinity of the radiopharmaceutical for Aβ-aggregates, and the health of the individual. The waiting time typically precedes any measurement of gamma radiation count. However, in some embodiments, a measurement of the gamma radiation count, as provided in step (c), begins immediately after administration of the Aβ-binding radiopharmaceutical. In this case, the waiting time is the instance between administration and the time when initial measurements are taken. These initial measurements may optionally be used to calculate the final gamma radiation count.

In some embodiments, the waiting time is from about 5 minutes to a time corresponding to approximately twice the radioactive half-life of the radioactive isotope of the Aβ-binding radiopharmaceutical. In other embodiments, the waiting time is from about 10 minutes to about a time corresponding to the radioactive half-life of the radioactive isotope of the Aβ-binding radiopharmaceutical. For example, in various aspects of the invention, the waiting time is from about 0.1 hour to about 24 hours, from about 0.1 hour to about 12 hours, from about 0.1 hour to about 6 hours, from about 0.1 hour to about 2 hours, or from about 0.1 hour to about 60 minutes. In certain embodiments, the waiting time is about 1 minute to about 60 minutes. Considerations such as patient convenience may make it preferable in some embodiments to perform measurements within one hour from the time of administration.

Step (c) of the method embodied above includes measuring a gamma radiation count over an external area of the head corresponding to the cortex of the individual using a radiation detection device. The gamma radiation count rate of various embodiments may be a radiation count rate or, alternatively, a total radiation count measurement on the surface of the cranium over a short period of time following the administration of the Aβ-aggregate binding radiopharmaceutical.

The gamma emission measurements are generally made over the cortex of the brain. For example, in some embodiments, the external measurement may be taken over the orbital frontal cortex on the side of an individual's head. The gamma emission measurements may be preceded, followed by or simultaneous with obtaining measurements over a portion of the individual's head that should not include amyloid plaque such as, for example, the cerebellum on the side of the back of the head, for example, posterior to and about even with the middle of the ear, or obtaining a measurements from healthy individuals. Such measurements are taken to obtain a control gamma radiation count.

The present invention is not limited to any one particular radiation detection device and any alternatives devised by one skilled in the art are encompassed by the invention. In certain embodiments, the radiation detection device includes some of the following components: a detector component (e.g., scintillation crystal) for capturing gamma rays and converting collected signal to a light or electrical impulse; a signal amplification stage, which may comprise a photomultiplier tube or series of photodiode amplifiers; an electronic circuit for filtering background noise and for amplification of the signal from the radioactive disintegrations detected; an integrator for summing the number of gamma rays detected; and a rate meter that measures the rate of radioactive disintegrations detected. In some embodiments, the radiation detection device excludes three-dimensional imaging techniques.

In some embodiments, the radiation detection device includes a detector component, which provides a detector component output, and a ratemeter, scaler, or integrator for measuring the gamma radiation count. In some embodiments, the detector component comprises a scintillation crystal and a detector output amplification component selected from one or more photomultiplier tubes, one or more photodiode amplifiers, and combination thereof. In various embodiments of the invention, the radiation detection device is of sufficient sensitivity to detect relatively small quantities of radioactivity in the brain. In some embodiments, the radiation detection device is portable. In sonic embodiments, the radiation detection device is stationary.

In other embodiments, the radiation detection device further includes a gamma radiation shield with a collimating aperture positioned between a detector component and an external area of an individual's head corresponding to the cortex. The gamma radiation shield with a collimating aperture may comprise, for example, lead or tungsten, in other embodiments of the invention, the radiation detection device further comprises a circuit for amplifying detector component output. In further embodiments, the radiation detection device includes a circuit for filtering background noise from the detector component output. The circuit may also amplify electrical impulses after the filtering out of background noise.

Step (d) of embodiments of the present invention includes comparing the gamma radiation count detected in step (c) with a control gamma radiation count. Step (d) may be carried out in a number of ways such as, for example, comparing the gamma radiation count over an external area of the head corresponding to the cortex of the individual with a gamma radiation count of a control region of the brain of the individual. Alternatively, step (d) may be performed by comparing the gamma radiation count over an external area of the head corresponding to the cortex of the individual at risk of having Aβ plaque with the same measurement taken in a healthy control population.

In certain embodiments, the control gamma radiation count may be obtained by measuring the gamma radiation count using a radiation detection device over an external area of the head corresponding to the frontal cortex of a healthy individual. In another embodiment, a control gamma radiation count that is an average or median gamma radiation count is obtained by measuring the gamma radiation count over an external area of a head corresponding to the cortex of a healthy individual using a radiation detection device, repeating, the measurement for a population of healthy individuals, and averaging or calculating the median counts for the control population. In still other embodiments, the control gamma radiation count is an average gamma radiation count obtained by measuring the gamma radiation count over an external area of a head corresponding to the cerebellum of an individual using a radiation detection device, repeating the measurement for a population of individuals, and averaging the counts for the control population.

The cortical measurement of radiation counts in an individual at elevated risk for or actually having AD (e.g., having a relevant amount of Aβ plaques in the brain) is generally significantly greater than the cortical measurement of radiation counts in a healthy individual. In addition, the ratio of radiation counts in the frontal region compared to cerebellar region in an individual is significantly greater where the individual is at elevated risk for or actually has AD compared to the ratio achieved in a healthy individual.

In some embodiments, the comparison of step (d) involves calculating the ratio of the cortical gamma radiation count for the individual measured in step (c) to the control gamma radiation count. A higher ratio would be consistent with the presence of amyloid peptide aggregates in the brain of the individual. In sonic embodiments, a ratio of above about 1.4 is consistent with a higher risk for AD or other neurodegenerative disorder and a ratio of about 1.4 or below is indicative of a lower risk. A lower ratio is consistent with not having a substantial amount of amyloid plaques in the brain. A higher ratio may indicate a substantial amount of amyloid plaques in the brain.

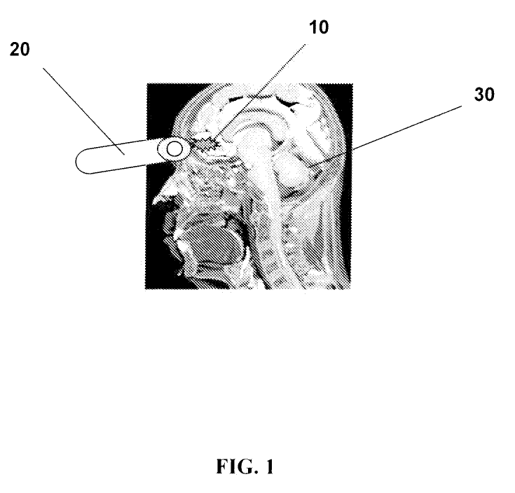

FIG. 1 schematically illustrates gamma probe detection of amyloid plaques following injection of radiolabeled Aβ-binding compounds according to one embodiment of the present invention. As shown in FIG. 1, Aβ-aggregates 10 in the orbital frontal region of a human brain are labeled with a gamma-emitting Aβ-aggregate binding radiopharmaceutical after a sufficient waiting time following administration of the Aβ-binding radiopharmaceutical. A gamma radiation count is measured over an external area of the head corresponding to the orbital frontal region using a portable gamma detector probe with shielded collimator 20. The gamma dectector probe 20 further comprises an amplifier and filter circuitry as well as an integrator/counter. The measured gamma radiation count is then compared with a control gamma radiation count. The control gamma radiation count is measured over an external area of the head corresponding to the cerebellum as a reference/control region using the portable gamma detector probe 20.

In some embodiments, the method for detecting β-amyloid peptide aggregates in the brain of an individual may he used to estimate the individual's relative risk of developing Alzheimer's disease (AD). In other embodiments, the method may be used to evaluate the progression of AD in the individual. In still other embodiments, the method may be used to diagnose Alzheimer's disease in the individual or to rule out the presence of Alzheimer's disease.

The Aβ-binding radiopharmaceuticals of embodiments of the present invention facilitate gamma probe measurement outside the cranium in a low-cost detection method for identifying amyloid plaque in the brain and consequently identifying individuals at elevated risk of having or developing AD or other neurodegenerative disorder. The Aβ-binding radiopharmaceutical utilized in embodiments of the present invention preferentially exhibits a high affinity for Aβ-aggregates. For example, in some embodiments, the affinity (e.g. K_{d}) of the Aβ-binding radiopharmaceutical is less than or equal to about 100 nM. In other embodiments, the binding affinity is about 10 nM or less.

The Aβ-binding radiopharmaceutical of various embodiments of the present invention includes a compound that selectively binds to Aβ aggregates, which is tethered to a radioactive particle or radiolabeled by any of numerous methods known in the art. In certain embodiments, the Aβ-binding pharmaceutical includes a radiolabeled antibody, protein, peptide, nucleic acid, organic melecule, polymer or a combination thereof. Specifically, in certain embodiments, isotopes within the Aβ-binding radiopharmaceuticals emit gamma rays of sufficient energy to traverse through brain tissue and be detected with an external radiation detection device.

A variety of radioisotopes may be attached to the Aβ-binding radiopharmaceutical for localization to amyloid plaques such as, but not limited to, ⁷⁶Br, ¹²³I, ¹²⁵I, ¹³¹I, ^{99m}Tc, ¹¹C, and ¹⁸F or a combination thereof. The radioisotopes useful in aspects of the present invention decay with an emission of gamma-rays detectable using external probe detection methodology (i.e, measurement taken outside of an individual's skull). In other embodiments, an Aβ-binding radiopharmaceutical may be identified using binding assays known in the art. In other embodiments of the present invention, a slightly modified assay can be used.

In particular embodiments, the radiolabeled compounds include ¹⁸F because of the specific decay half-life provided (approximately 110 minutes), which allows relatively rapid decay of the radiopharmaceutical in the patient after the probe measurements are completed thereby allowing the subject to safely return to work or home, but the decay half-life is not so short as to cause a major loss of signal in the brain prior to adequate blood clearance over the first 30-60 minutes after injection.

The Aβ-binding radiopharmaceutical may contain one or more asymmetric centers, which can give rise to optical isomers (enantiomers) and diastereomers. Hence, the Aβ-binding radiopharmaceutical can include an enantiomer, diastereomer, racemate or mixtures thereof of the Aβ-binding radiopharmaceutical. In some embodiments, the Aβ-binding radiopharmaceutical exists as a geometrical isomer. In addition, the present invention encompasses all possible regioisomers and mixtures thereof, which can be obtained in pure form by standard separation procedures known to those skilled in the art, such as, for example, column chromatography, thin-layer chromatography, and high-performance liquid chromatography. Tautomers for the Aβ-binding radiopharmaceutical are also encompassed in embodiments of the present invention.

Examples of the Aβ-binding radiopharmaceuticals in embodiments of the present invention include, but are not limited to, those described in WO 2006/014381 (PCT/US/2005/023617), U.S. 2003/0236391 (Ser. No. 10/388,173), U.S. 2005/0043523 (Ser. No, 10/645,847), WO 2007/047204 (PCT/US2006/039412), WO 2007/086800 (PCT/SE2007/000068), WO 2006/057323, EP 1815872 (PCT/JP2005/021642), WO 2005/016888, EP 1655287 (PCT/JP04/11546), U.S. Pat. No. 6,696,039, U.S. Pat. No. 6,946,116, U.S. Pat. No. 7,250,525, WO 2006/078384 (PCT/US2005/045683), WO 2006/066104 (PCT/US2005/045682), WO 2007/126733 (PCT/US2007/007400), U.S. 2006/269473, U.S. 2006/269474, U.S. 2005/0271584, U.S. 2007/0031328, Mathis et al., J Med. Chem. 2003, 46:2740-2754: Small et al., N Engl. J. Med. 2006, 355:2652-2663: Zhang et al., Nucl. Med. Biol., 2005, 32:799-809; Ono et al., Nucl. Med. Biol. 2002, 29:633-642; Ono et al., Nucl. Med. Biol. 2005, 32:329-335; Qu et al., Bioinorg. Med. Chem. Lett. 2007, 17:3581-3584; Kemppainen et al., Neurology 2007, 68:1603-1606, Pike et al., Brain 2007, 130; 2837-2844; Klunk et al. Ann. Neurol. 2004; 55, 306-319; Verhoeff et al., Am J Geriatr Psychiatry 2004; 12, 584-595; and Newberg et al., J Nucl Med. 2006; 47, 748-754, each of which is hereby incorporated by reference in its entirety.

The half-life of the Aβ-binding radiopharmaceutical of embodiments of the present invention may vary depending on which radioisotope is utilized. Accordingly, in some embodiments, the Aβ-binding radiopharmaceutical has a radioactive half-life of about 24 hours or less. In other embodiments, the radioactive half-life of the Aβ-binding radiopharmaceutical may be about 12 hours or less, in still others, about 6 hours or less, and in some, about 2 hours to about 1 hour or less.

The radioactivity emitted by the Aβ-binding radiopharmaceutical may vary among embodiments, and may depend upon various aspects of the procedure (i,e., the waiting period) or the physiology of the individual. As such, the amount of Aβ-binding radiopharmaceutical administered can vary among embodiments, as does the effective amount of the Aβ binding radiopharmaceutical. For example, in some embodiments 0.1 to 20 mCi (3.7 to 740 MBq) of the Aβ-binding radiopharmaceutical is administered to the individual. In this case, an effective amount may be from about 0.1 to about 20 mCi of the Aβ-binding radiopharmaceutical. In other embodiments, the effective amount of the Aβ-binding radiopharmaceutical may be from about 0.1 to about 10 mCi. In still other embodiments, the effective amount may be from about 0.1 to about 2 mCi. In further embodiments, lower doses of Aβ-binding radiopharmaceutical may be administered and function as an effective amount.

The Aβ-binding radiopharmaceutical may be administered by any method of administration. For example, in some embodiments, the Aβ-binding radiopharmaceutical is administered orally, rectally, parenterally (e.g., intravenous, intramuscularly or subcutaneously), intracistemally, intravaginally, intraperitoneally, intravesically, or locally as, for example, powders, ointments or drops, or as a buccal or nasal spray. In preferred embodiments, the Aβ-aggregate-binding radiopharmaceutical is administrated by injection, and more preferably may be administered by intravenous injection. Aβ-aggregate binding radiopharmaceuticals useful in embodiments of the invention can be administered in a pharmaceutical composition in unit dosage form. For example, Aβ-aggregate binding radiopharmaceuticals formulated for intravenous administration may be prepared in unit dose syringes containing an appropriate quantity of active ingredient.

The individual in various embodiments being measured for the presence of Aβ amyloid plaque in the brain may be any living creature. For example, in some embodiments, the individual is a mammal and preferably a living human being. In certain embodiments, the individual may be at risk for developing amyloid plaque and/or Alzheimer's disease or suspected of having Alzheimer's disease, and in particular embodiments, the individual may be an elderly individual.

More specific embodiments of the invention are provided below. These embodiments are not meant to be limiting. It is appreciated that certain features of the invention, which are, for clarity, described in the context of separate embodiments, can also be provided in combination in a single embodiment. Conversely, various features of the invention that are, for brevity, described in the context of a single embodiment, can also be provided separately or in any suitable subcombination. The elements and steps described herein may be combined in any number of ways as determined by the skilled artisan to affect a desired outcome without deviating from the spirit and scope of the invention. For example, in some embodiments, the effective amount of Aβ-binding radiopharmaceutical includes from about 0.1 to about 20 mCi of a radioisotope wherein the radiopharmaceutical is a compound having a binding affinity of ≦100 nM for Aβ-aggregates and is labeled with one or more radioisotopes having a radioactive half-life of about 24 hours or less such as, for example, ¹¹C, ¹⁸F, ^{99m}Tc, ¹²³I, or combination thereof. In this embodiment, the waiting time is from about 0.1 hour to a time corresponding to about the radioactive half-life of the radioisotope attached to the Aβ-binding radiopharmaceutical. Additionally, in this embodiment, the control gamma radiation count is obtained by measuring the gamma radiation over an external area of the head corresponding to the cerebellum region of the individual using the radiation detection device or measuring the gamma radiation over the external area of the head of a healthy individual.

In other embodiments, the effective amount of Aβ-binding radiopharmaceutical includes from about 0.1 to about 10 mCi of a radioisotope; the Aβ-binding radiopharmaceutical is a compound that is radiolabeled with ¹¹C, ¹⁸F, ^{99m}Tc, ¹²³I, or combination thereof; the waiting time is from about 0.1 hours to a time corresponding to about the half-life of a radioisotope attached to the Aβ-binding radiopharmaceutical; and the control gamma radiation count is obtained by measuring the gamma radiation over the external area of the head corresponding to the cerebellum of an individual.

In still other embodiments, the effective amount of Aβ-binding radiopharmaceutical includes from about 0.1 to about 2 mCi of a radioisotope; the Aβ-binding radiopharmaceutical is a compound that is radiolabeled with ¹¹C, ¹⁸F, ^{99m}Tc, ¹²³I, or combination thereof; the waiting time is from about 0.1 hours to a time corresponding to about the hall-life of a radioisotope attached, to the Aβ-binding radiopharmaceutical; and the control gamma radiation count is obtained by measuring the gamma radiation over the external area of the head corresponding to the cerebellum of an individual.

In other embodiments of the present invention, the effective amount of Aβ-binding radiopharmaceutical includes from about 0.1 to about 20 mCi of a radioisotope; the Aβ-binding radiopharmaceutical is a compound radiolabeled with ¹⁸F; the waiting time is from about 0.1 hours to a time corresponding to about the half-life of ¹⁸F; and the control gamma radiation count is obtained by measuring the gamma radiation over the external area of the head corresponding to the cerebellum of an individual.

In still other embodiments, the effective amount of Aβ-binding radiopharmaceutical includes from about 0.1 to about 10 mCi of a radioisotope; the Aβ-binding radiopharmaceutical is a compound radiolabeled with ¹⁸F; the waiting time is from about 0.1 hours to a time corresponding to about the half-life of ¹⁸F; and the control gamma radiation count is obtained by measuring the gamma radiation over the external area of the head corresponding to the cerebellum of the individual.

In yet other embodiments, the effective amount of Aβ-binding radiopharmaceutical includes from about 0.1 to about 2 mCi of a radioisotope; the Aβ-binding radiopharmaceutical is a compound radiolabeled with ¹⁸F; the waiting time is from about 0.1 hours to a time corresponding to about the half-life of ¹⁸F; and the control gamma radiation count is obtained by measuring the gamma radiation over the external area of the head corresponding to the cerebellum of an individual.

In some embodiments, the effective amount of Aβ-binding radiopharmaceutical comprises from about 0.1 to about 20 mCi of a radioisotope; the waiting time is from about 0.1 hours to about 6 hours; and the control gamma radiation count is obtained by measuring the gamma radiation over the external area of the head corresponding to the cerebellum of an individual.

In other embodiments, the effective amount of Aβ-binding radiopharmaceutical includes from about 0.1 to about 20 mCi of a radioisotope; the waiting time is from about 0.1 hours to about 2 hours; and the control gamma radiation count is obtained by measuring the gamma radiation over the external area of the head corresponding to the cerebellum of an individual.

In still other embodiments, the effective amount of Aβ-binding radiopharmaceutical includes from about 0.1 to about 20 mCi of a radioisotope; the waiting time is from about 10 minutes to about 60 minutes and the control gamma radiation count is obtained by measuring the gamma radiation over the external area of the head corresponding to the cerebellum of an individual.

In further embodiments, the effective amount of Aβ-binding radiopharmaceutical includes from about 0.1 to about 10 mCi of a radioisotope; the waiting time is from about 0.1 hours to about 6 hours; and the control gamma radiation count is obtained by measuring the gamma radiation over the external area of the head corresponding to the cerebellum of an individual.

In some embodiments, the effective amount of Aβ-binding radiopharmaceutical includes from about 0.1 to about 10 mCi of a radioisotope; the waiting time is from about 0.1 hours to about 2 hours; and the control gamma radiation count is obtained by measuring the gamma radiation over the external area of the head corresponding to the cerebellum of an individual.

In other embodiments, the effective amount of Aβ-binding radiopharmaceutical includes from about 0.1 to about 10 mCi of a radioisotope; the waiting time is from about 10 minutes to about 60 minutes; and the control gamma radiation count is obtained by measuring the gamma radiation over the external area of the head corresponding to the cerebellum of an individual.

In still other embodiments, the effective amount of Aβ-binding radiopharmaceutical includes from about 0.1 to about 2 mCi of a radioisotope; the waiting time is from about 0.1 hours to about 6 hours; and the control gamma radiation count is obtained by measuring the gamma radiation over the external area of the head corresponding to the cerebellum of an individual.

In yet other embodiments, effective amount of Aβ-binding radiopharmaceutical includes from about 0.1 to about 2 mCi of a radioisotope, the waiting time is from about 0.1 hours to about 2 hours; and the control gamma radiation count is obtained by measuring the gamma radiation over the external area of the head corresponding to the cerebellum of an individual.

In some embodiments, the effective amount of Aβ-binding radiopharmaceutical comprises from about 0.1 to about 2 mCi of a radioistotope; the waiting time is from about 10 minutes to about 60 minutes; and the control gamma radiation count is obtained by measuring the gamma radiation over the external area of the head corresponding to the cerebellum of an individual.

Embodiments of the invention further include a kit for detecting amyloid plaques in the brain of an individual. The kit may generally include an Aβ-binding radiopharmaceutical along with instructions directed to administering the Aβ-binding radiopharmaceutical to an individual, waiting for a period of time, and measuring the gamma radiation count over an external area of the head corresponding to the cortex of the individual using the radiation detection device. In certain embodiments, the Aβ-binding radiopharmaceutical is provided in dosage form for intraveneous injection. The instructions may further comprise a direction to compare the gamma radiation count with a control gamma radiation count, and in certain embodiments, the instructions may further include directions for measuring the gamma radiation count over the external area of the head away from the cortex such as, for example, an area corresponding to the cerebellum of the individual and comparing the gamma radiation count with a control gamma radiation count. In other embodiments, the kit further includes a radiation detection device and instructions for using such radiation detection device. In still other embodiments, the kit includes instructions for using radiation detection devices at a disclosed location. The kits in various aspects or the present invention may be used according to any of the methods embodied herein.

### EXAMPLES

In order that the invention disclosed herein may be more efficiently understood, examples are provided. The following examples are for illustrative purposes only and are not to be construed as limiting the invention m any manner.

### Example 1

To demonstrate the feasibility of one embodiment of the present invention for detecting the quantities of radioactivity present in the brain of an Alzheimer's disease (AD) subject, AD subjects were injected with approximately 10 mCi of an ¹⁸F radiolabeled (E)-4-(2-(6-(2-(2-(2)-fluoro(18)-ethoxy)ethoxy)ethoxy)pyridin-3-yl)vinyl)-N-methylbenzenamine, the structure of which is shown in Example 1.

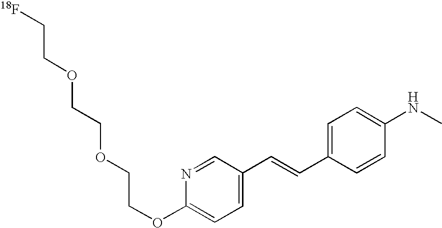

### Example 1

Positron emission tomography (PET) scans of the brain of the AD subjects were taken and analyzed. The amount of ¹⁸F in the frontal cortical regions of the respective AD subjects was characterized based on the number of radioactive counts per voxel per minute from the PET image. This data was converted to the units of kBq/cc of brain tissue in the frontal cortex. This was determined to be between 2.1 to 2.9 kBq/cc (with some variation between subjects and voxels sampled in the frontal cortex). In addition, the PET images were analyzed to determine the approximate amount of ¹⁸F Aβ-binding compound in the cerebellum, a reference region of the brain with little or no amyloid plaques. The amount of radioactivity in the cerebellum ranged from 1.0 to 1.5 kBq/cc.

Based on these amounts of ¹⁸F Aβ-binding compound in the frontal region of the brain (where amyloid plaques are typically found in AD patients) versus the cerebellum, two beakers of water with approximately 100 cc each (mimicking the volumes of the frontal and cerebellar regions of the brain) were prepared with 0.05μCi/cc (approximately 2 kBq/cc) and 0.025μCi/cc (approximately 1 kBq/cc) of ¹⁸F in each.

A general purpose gamma radiation survey meter (Ludlum Measurements, Inc., Model 3 Survey Meter) equipped with a gamma radiation probe detector (Ludlum Measurements, Inc., Model 44-38) was utilized. A lead shield of approximately 1 cm thickness with a 10 mm collimator opening was positioned over the end of the detector and measurements of the gamma radiation levels were made with the 10 mm collimator shield opening adjacent to the surface of each beaker. The survey meter reading was 100 counts per minute (CPM) for the beaker containing 2 kBq/cc of ¹⁸F in solution and the meter reading was 50 CPM for the beaker containing 1 kBq/cc of ¹⁸F in solution. These measurements demonstrate the feasibility of using a gamma probe with collimation to detect levels of ¹⁸F similar to those levels found in the frontal and cerebellar brain regions of AD subjects injected with ¹⁸F radiolabeled amyloid plaque-binding compounds for PET scanning. Moreover, the measurements with the gamma probe having a lead collimator shielded opening demonstrate the feasibility of detecting an approximate 2-fold difference in signal from ¹⁸F levels, which approximates the signal difference between the frontal and cerebellar brain regions of AD subjects injected with a ¹⁸F radiolabeled amyloid plaque-binding compound.

### Example 2 (Prophetic)

An intravenous injection of 37 to 74 MBq of ¹⁸F radiolabeled (E)-4-(2-(6-(2-(2-(2-[¹⁸F]fluoroethoxy)ethoxy)ethoxy)pyridin-3-yl)vinyly-N-methylbenzen amine is administered to an individual. After a waiting time of 45 minutes, the side of the individual's head is positioned adjacent to the shielded collimator gamma detection probe, as described in Example 1, so that the probe detects the gamma radiation in the frontal-orbital cortical region of the brain. Alternatively, a NaI scintillation probe (Ludlum Measurements, Inc., Model 44-2) is utilized. A measurement of the total gamma radiation count is taken over a 60 second period. The gamma radiation count measurement is then repeated in a similar manner on the side of the back of the head over the cerebellar region. The radiation count ratio of frontal to cerebellar regions is indicative of the individual's relative risk of having Aβ aggregates in the brain. Specifically, a ratio of above approximately 1.4 is consistent with a higher risk and a ratio of below about 1.3 is indicative of a lower risk of the individual having Aβ aggregates in the brain.

Various modifications of the invention, in addition to those described herein, will be apparent to those skilled in the art from the foregoing description. Such modifications are intended to fall within the scope of the appended claims.

1. A method of detecting β-amyloid aggregates in a brain of an individual, comprising:
administering to an individual an effective amount of an Aβ-binding radiopharmaceutical;
waiting a period of time;
measuring a gamma radiation count over an external area of the head corresponding to the cortex of the individual using a radiation detection device; and
comparing the gamma radiation count with a control gamma radiation count.

2. The method of claim 1, wherein the effective amount comprises from about 0.1 to about 20 mCi of said Aβ-binding radiopharmaceutical.

3. The method of claim 1, wherein the effective amount comprises from about 0.1 to about 10 mCi of said Aβ-binding radiopharmaceutical.

4. The method of claim 1, wherein the effective amount comprises from about 0.1 to about 2 mCi of said Aβ-binding radiopharmaceutical.

5. The method of claim 1, wherein the Aβ-binding radiopharmaceutical comprises a compound having a binding affinity of ≦100 nM for Aβ-aggregates.

6. The method of claim 1, wherein the Aβ-binding radiopharmaceutical comprises a compound having a binding affinity of about 10 nM or less.

7. The method of claim 1, wherein the Aβ-binding radiopharmaceutical comprises ⁷⁶Br, ¹²³I, ¹²⁵I, ¹³¹I, ^{99m}Tc, ¹¹C or ¹⁸F.

8. The method of claim 1, wherein the period of waiting time is from about 5 minutes to a time corresponding to approximately twice the radioactive half-life of the radioactive isotope of the Aβ-binding radiopharmaceutical.

9. The method of claim 1, wherein the period of waiting time is from about 10 minutes to a time corresponding to the radioactive half-life of the radioactive isotope of the Aβ-binding radiopharmaceutical.

10. The method of claim 1, wherein the period of waiting time is about 1 to about 60 minutes.

11. The method of claim 1, wherein the control gamma radiation count is a gamma radiation count of a control region in the brain of the individual.

12. The method of claim 1, wherein the control gamma radiation count is a gamma radiation count obtained over an external area of the head corresponding to the cortex of individuals from a healthy control population.

13. The method of claim 1, wherein the control gamma radiation count is an average or median gamma radiation count determined by repeating the gamma radiation count measurement for a population of healthy individuals and calculating the average or median counts for the control population.

14. The method of claim 1, wherein the comparing step comprises calculating a ratio of the cortical gamma radiation count for an individual to the control gamma radiation count.

15. The method of claim 14, wherein the calculated ratio of above about 1.4 is consistent with the presence of β-amyloid peptide aggregates in the brain of the individual.

16. The method of claim 1, wherein
the effective amount comprises from about 0.1 to about 20 mCi of said Aβ-binding radiopharmaceutical;
the Aβ-binding radiopharmaceutical comprises a compound having a binding affinity of ≦100 nM for Aβ-aggregates;
the period of waiting time is from about 0.1 hours to a time corresponding to about the radioactive half-life of the radioisotope attached to said Aβ-binding radiopharmaceutical; and
the control gamma radiation count is obtained by measuring the gamma radiation over the external area of the head corresponding to the cerebellum region of said individual using said radiation detection device.

17. A kit for detecting amyloid plaques in the brain of an individual, comprising:
an Aβ-binding radiopharmaceutical; and
instructions for using the Aβ-binding radiopharmaceutical, said instructions comprising a direction to administer to an individual an effective amount of an Aβ-binding radiopharmaceutical; a direction to wait a period of time; and a direction to measure a gamma radiation count over an external area of the head corresponding to the cortex of said individual using a radiation detection device.

18. The kit of claim 17, wherein the Aβ-binding radiopharmaceutical is in dosage form for intravenous injection.

19. The kit of claim 17, further comprising a radiation detection device and instructions for using such radiation detection device.

20. The kit of claim 17, further comprising instructions for using a radiation detection device at a disclosed location.
